(19) United States
(12) Patent Application Publication
(10) Pub. No.: US 2014/0351966 A1
(43) Pub. Date: Nov. 27, 2014

(54) SHORTCUT PROCEDURE OF TRANSGENE INTEGRATION BY HYPOTONIC SHOCK INTO MALE GERMINAL CELLS FOR GENE EXPRESSION AND TRANSGENESIS

(71) Applicant: National Institute of Immunology, New Delhi (IN)

(72) Inventors: Subeer Suhash Majumdar, New Delhi (IN); Abul Usmani, New Delhi (IN); Nirmalya Ganguli, New Delhi (IN)

(73) Assignee: National Institute of Immunology, New Delhi (IN)

(21) Appl. No.: 14/096,634

(22) Filed: Dec. 4, 2013

(30) Foreign Application Priority Data
Dec. 11, 2012 (IN) 3799/DEL/2012

(51) Int. Cl. A01K 67/027; A61D 19/02

(52) U.S. Cl. CPC A01K 67/0275; A61D 19/02; A01K 2227/105
USPC 800/21; 435/455; 600/35

(57) ABSTRACT

The present invention relates to a new method of gene integration in cells comprising the steps of: preparation of a suspension of poly nucleotide fragments comprising the desired gene in a hypotonic solution; administration of said suspension to cells or tissues; and maintenance of the cells or tissues in vitro or in its normal physiological condition. The invention also relates to a method of generating transgenic animal.

### RELATED APPLICATION

This application claims the benefit of priority, under 35 U.S.C. Section 119, to India Patent Application Serial No. 3799/DEL/2012, filed Dec. 11, 2012, which is incorporated herein by reference in its entirety.

### FIELD OF THE INVENTION

The present invention relates to a new method of transgenesis. More particularly, the present invention relates to a quick method of delivering nucleic acid fragments or transgene or gene of interest into vertebrate male germ cells, specifically spermatogonial stem cells (SSCs), by breaching the cell membrane using hypotonic solution to deliver poly nucleotide fragments inside the cells.

### BACKGROUND OF THE INVENTION

Gene delivery is a process which includes delivery of nucleic acid materials in the eukaryotic cells. This is achieved using viral transduction or various physical method or by treating the cells with various chemical along with nucleic acid materials. Delivery of gene through these various processes is performed either in-vitro or in-vivo to study or evaluate the role of those genes in the context of cellular function or in actual physiological condition. Gene therapy is also a prime target of in-vivo or in-vitro nucleic acid delivery.

There are various gene delivery methods known. However, the main factor is to overcome the challenges related to the issue of introducing negatively charged polynucleotide fragments (like deoxyribonucleic acid-DNA and ribonucleic acid-RNA) into cells through a negatively charged membrane leading to inefficient delivery or gene expression and causing mortality of cells or disruption of tissue architecture.

TABLE 1

| | |
|---|---|
| Chemical Methods | Calcium phosphate, highly branched organic |
| | compounds (dendrimers), liposomes, cationic |
| | polymers. |
| Physical Methods | Sono-poration, Electroporation, Optical |
| | transfection, Impalefection, Hydrodynamic |
| | delivery, gene gun, Magnetofection, heat |
| | shock, naked DNA transfer. |
| Viral Method | Adeno viral transduction, Lentiviral |
| | transduction. |

The limiting factors are efficiency of gene integration and breaching of cellular function due to prolonged time required for such procedures.

Researchers have been for a long time using the various techniques listed in Table 1 to deliver DNA sequences coding for gene in the male germ cells or in other somatic cells of body for generating transgenic animal or for gene therapy purpose. Vertebrate male germ cells have enormous potential for generating transgenic animals. There is a big demand for evaluating the role of various nucleic acid sequences (functional genomics) which are identified as a gene by the use of various high throughput techniques but their functions are not known. In vitro or in vivo integration and expression of gene can provide substantial information about their functional role.

Although it has been shown previously that vertebrate male germ cells can incorporate and propagate foreign gene; the approach is labor intensive and time consuming. It also involves surgical procedures and killing of animals. In in vitro approach, the male germ cells obtained from the testis are manipulated/transfected in vitro. In case of germ cells or crude testicular suspensions, they are surgically transplanted into testis of a recipient male after manipulation.

Brackett et al. (Proc. Natl. Acad. Sci. USA, 1971, 68, 353-357) provided the first report that the foreign DNA can be introduced into sperm. This group showed that DNA labeled with tritiated thymidine could go inside the head of rabbit spermatozoa when incubated in toto.

Almost 20 years later, Lavitrano et al. (Cell, 1989, 57, 717-723) showed that circular or linear DNA could be incorporated into mouse epididymal spermatozoa by simple incubation.

Kroll and Amaya (Development, 1996, 122, 3173-3183) tried to introduce linearized plasmid DNA into the nuclei of the Xenopus sp. sperms using restriction enzyme-mediated integration (REMI) to decondense the genomic DNA. Shemesh et al., (Mol. Reprod. Dev., 2000, 56, 306-308) combined this approach with lipofection to integrate the transgene into the genomic DNA of the sperm before IVF and applied to cattle with partial success. However, the major problem associated with these different approaches is that the treatments used to help the exogenous DNA cross the natural sperm barrier during transfection also results in killing of spermatozoa.

Akutsu et al. (US 2004/0210955 A1) discloses methods of preparing spermatozoa for use in ICSI-mediated transgenesis. However, in this process the spermatozoon is treated in a medium comprising a chelating agent and then incubated with an exogenous nucleic acid for a period of time.

Wakayama and Yanagimachi (Nat. Biotechnol. 1998, 16, 639-641) and Perry et al. (Science, 1999, 284, 1180-1183) tried to overcome this problem and subjected spermatozoa to Triton X-100 treatment, repeated freeze-thaw cycles or freeze-drying cycles before incubation with exogenous DNA. However, this again resulted in non-motile or dead sperms incapable of fertilization.

Perry (US 2004/0088748 A1) discloses method of generating transgenic animals by co-inserting nucleic acid and a nucleus by microinjection into an unfertilized oocyte. However, like in the above stated paragraph, the sperm nuclei were subjected to freeze thaw, or freeze drying or triton X-100 extraction which resulted in sperm damage.

Wakayama and Yanagimachi (Nat. Biotechnol. 1998, 16, 639-641) injected sperm exposed to DNA, into the cytoplasm of the oocyte using costly and complicated micromanipulator assembly followed by artificial stimulus for cell division. Even though these sperm mediated approaches are simple they have not been reproducible. In most of the cases, the transgene remains episomal and the expression is lost as the animal grows. Further, these approaches require the use of costly and difficult-to-practice assisted reproductive techniques such as ICSI and embryo transfer for generation of founders, which requires superovulation and killing of animals for egg retrieval.

The most recent approach developed is electroporation based. Yamazaki et al. (Biol. Reprod., 1998, 59, 1439-1444) injected DNA into testicular seminiferous tubules followed by electroporation. This resulted in long-term transgene expression in the injected testes (>2 months). Yamazaki et al. (J. Exp. Zool., 2000, 286, 212-218) were, however, unable to obtain any transgenic offspring.

Huang et al. (FEBS Lett., 2000, 487, 248-251) failed to get fluorescent spermatozoa using Green fluorescent protein (GFP) in another electroporation study. When Yellow fluorescent protein (YFP)-based construct was used instead of GFP for electroporation, fluorescence was seen, but the expression did not last long.

Yomogida et al. (Biol. Reprod., 2002, 67, 712-717) in their study found that the transgene upon testicular electroporation was not integrated into the germ line and was dominantly expressed in Sertoli cells.

Umemoto et al. (J. Androl., 2005, 26, 264-271) transferred pCAGGS-lacZ to mouse testes using electroporation. However, none of the offspring carried the transgene when these male mice were mated with wild type females and the progeny was screened for lacZ.

Harvey et al. (US 2004/0259130 A1) describes methods of producing an integrated transgene in an avian cell by introducing a nucleic acid into an avian cell by electroporation. It particularly relates to producing a transgene avian by injecting the cell containing the transgene into stage X embryo.

In addition to the above non confirmatory methods, a more efficient method of transgenesis has been established recently. Dhup and Majumdar (Nature Methods., 2008, 5(7), 601-603) established permanent integration of genes into Spermatogonial Stem Cells (SSCs) by application of electric pulses after insertion of genes in the testis of mice following surgical incision. In this procedure, testis was surgically exposed under the stereo zoom microscope in sterile environment and linearized DNA was directly injected into one of the testis. This was followed by electroporation with square wave pulses of 50 volts for 40 milliseconds in forward (4 pulses) and reverse (4 pulses) direction. After the electric pulses, the testis was inserted in capsule followed by suturing of the capsule and the skin. One of the major drawbacks was that in this procedure, contralateral testis was removed by castration. In spite of high efficiency, this method is not widely used. The various surgical steps involved in this procedure increases the chances of pain, post-surgical infection and impotency of male mice. Only a skilled person who is trained in reproductive biology and surgery could perform the procedure well and with ease.

The other technique of transgenesis includes traditional, male pronuclear DNA microinjection, retroviral mediated method and embryonic stem (ES) cell-mediated method. Pronuclear DNA microinjection was the first developed method for gene insertion in animals. Gordon et al., (Proc. Natl. Acad. Sci. U.S.A., 1980, 77, 7380-7384) led to the production of the first transgenic mice because of the development of micromanipulation techniques together with studies on the capacity of preimplantation of embryos to transcribe and translate exogenously introduced genetic material.

Hammer et al. (Nature. 1985, 315: 680-683); Ebert et al. (Bio/Technology, 1991, 9, 835-838); Bondioli et al. (First N, Haseltine F (ed), Transgenic Animals. London: Butterworth-Heinemann. 1991, pp 265-272); Hill et al. (Theriogenology. 1992, 37, 222-222) subsequently applied this technology successfully to various other species such as rabbit, sheep, pig, goat and cattle. Even though this method is effective and successful, it is still cumbersome to perform and requires hundreds of eggs from several super ovulated females as well as costly infrastructure which proved a major hindrance in its establishment. Jaenisch (Proc. Natl. Acad. Sci. U.S.A., 1976, 73, 1260-1264) used retrovirus-mediated transgenesis and Gossler et al. (Proc. Natl. Acad. Sci. U.S.A., 1986, 83, 9065-9069) used embryonic stem (ES) cell-mediated gene transfer to generate transgenic animals.

All the above procedures are suitable for use in laboratory mice or rats, but difficult for use in large animals. Surgical procedure in the scrotal area may not be suitable for use in large farm animals that are prone to infection due to non-hygienic conditions.

### SUMMARY OF THE INVENTION

It is an object of the present invention to provide a simple, effective and short cut method of gene transfer in spermatogonial cells.

A further object of the invention is to provide a method for generation of transgenic animals.

Yet another object of the invention is to provide use of this method for gene therapy in germ cells of the treated recipient and permanent rectification of the defect by eliminating the ill effect in future progeny sired by such recipient.

Accordingly, the present invention provides a method of delivering polynucleotide fragments or transgene or gene of interest or therapeutic agent inside cell.

In an embodiment, the method of the present invention comprises a simple hypotonic solution which selectively causes temporary opening up of male germ cell membrane and facilitate transgenesis.

In one embodiment, the method of the present invention comprises administration of hypotonic solution along with fragments of poly nucleotides which delivers desired gene into testicular spermatogonial stem cells.

In another embodiment of the invention, the method of gene expression and transgenesis comprises the steps of: preparation of a gene construct comprising the desired polynucleotide fragments and cloning; isolation and purification of desired poly nucleotide fragments; preparation of a suspension of desired poly nucleotide fragments in a hypotonic solution of neutral pH; administration of the suspension to the vertebrate cells or tissues; maintenance of the vertebrate cells or tissues in culture condition or in its normal physiological condition; and detection and/or utilization of cells containing the desired poly nucleotide fragments.

In a further embodiment of the invention, said hypotonic solution is Tris-HCl solution of neutral pH.

In another embodiment of the invention, said hypotonic solution is 150 mM Tris-HCl.

In one embodiment of the invention, said vertebrate cells comprise male germ cells, other cell types of the vertebrate male testis, female germ cells, and/or somatic cells.

In another embodiment of the invention, said germ cells comprise male and female germ cells.

In another embodiment of the invention, said method is performed in-vitro as well as in-vivo.

In yet another embodiment of the invention, said poly nucleotides comprise of DNA, RNA, shRNA, siRNA, and/or miRNA.

Still another embodiment of the invention is a method of generating a transgenic animal comprising the steps of: preparation of a gene construct comprising the desired polynucleotide fragments and cloning; isolation and purification of desired poly nucleotide fragments; preparing a suspension of desired poly nucleotide fragments in a hypotonic solution; administration of said suspension to the vertebrate cells or tissues; maintenance of the vertebrate cells or tissues in its normal physiological condition; detection of cells containing the desired poly nucleotide fragments; and utilization of cells containing the desired poly nucleotide fragments in generating transgenic animals by natural mating or by assisted reproductive technique (ART).

In an embodiment of the invention, said hypotonic solution is 150 mM Tris-HCl.

In another embodiment of the invention, said vertebrate cells are germ cells

In another embodiment of the invention, said germ cells comprise male and female germ cells.

In one embodiment of the invention, said ART comprises in vitro fertilization (IVF) and intra-cytoplasmic sperm injection (ICSI).

Use of the method of the invention in generation of transgenic animals of commercial importance and use them as a bioreactor for production of biopharmaceuticals and/or nutraceuticals forms yet another embodiment of the invention.

Use of the method of the invention for gene therapy forms still another embodiment of the invention.

### BRIEF DESCRIPTION OF THE FIGURES

FIG. 1 is a schematic representation of the method and principle of polynucleotide fragment delivery in the cell through hypotonic solution.

FIGS. 2(a) to (b) depict in vitro cultured Seminiferous tubules observed under UV stereo zoom Microscope, 2 hours after treatment with fluorophore labelled Red dUTP suspended in various concentration of Tris-HCl (hypotonic solution). Left panel shows the image under UV and right panel shows the image under bright field.
a) shows image of seminiferous tubules treated with fluorophore labelled Red dUTP suspended in 50 mM Tris-HCl.
b) shows image of seminiferous tubules treated with fluorophore labelled Red dUTP suspended in 100 mM Tris-HCl. Arrow shows the tubule with fluorescence while the dotted arrow shows tubules which does not have fluorescence.

FIG. 3 depicts in vitro cultured Seminiferous tubules observed under UV stereo zoom microscope, 2 hours after treatment with fluorophore labelled Red dUTP suspended in 150 mM of Tris-HCl (hypotonic solution). Left panel shows the image under UV and right panel shows the image under bright field.

(a) to (b) shows two different image fields of seminiferous tubules treated with fluorophore labelled Red dUTP suspended in 150 mM Tris-HCl. Arrow shows the tubule with fluorescence while the dotted arrow shows tubules which does not have fluorescence.

FIGS. 4 (a) to (e) depict generation of desired fragment of pCX-EGFP and MIS-IRES2-EGFP constructs used in the method:
a) agarose Gel Electrophoresis picture of pCX-EGFP plasmid obtained after isolation.
b) digestion of pCX-EGFP plasmid was done by Sal I restriction enzyme. It generates single fragment of size ˜5.5 kb which contains the gene of interest (EGFP). 1 kb ladder from NEB was used for reference. Linearized fragment was eluted in Milli Q Distilled Water (MQ) from the gel, purified, dried under vacuum and redissolved in hypotonic solution. An aliquot was run on 1% TAE gel.
c) agarose gel electrophoresis picture of MIS-IRES2-EGFP plasmid obtained after plasmid isolation.
d) digestion of MIS-IRES2-EGFP plasmid was done in large scale by Ase I and Nhe I restriction enzyme. It generates two fragments of size ˜5.3 kb, and ˜592 bp. 1 kb ladder from NEB was used for reference. 5.3 kb fragment contains the gene of interest (EGFP).
e) 5.3 kb fragment was eluted in MQ from the gel, purified, dried under vacuum and redissolved in hypotonic solution. An aliquot was run on 1% TAE gel.

FIGS. 5(a) to (c) depict immuno-histochemical analysis for detection of EGFP in in-vitro cultured seminiferous tubule, treated with linearized pCX-EGFP plasmid suspended in various concentration of hypotonic solution of Tris-HCl (2.5 mM, 5 mM, 10 mM). Left panel shows the image under UV and right panel shows the image under bright field.
a) shows the image of seminiferous tubule treated with linearized pCX-EGFP plasmid suspended in 2.5 mM Tris-HCl solution.
b) shows the image of seminiferous tubule treated with linearized pCX-EGFP plasmid suspended in 5 mM Tris-HCl solution.
c) shows the image of seminiferous tubule treated with linearized pCX-EGFP plasmid suspended in 10 mM Tris-HCl solution.

FIGS. 6(a) to (c) depict immuno-histochemical analysis of seminiferous tubule for detection of EGFP, treated with linearized pCX-EGFP plasmid suspended in various concentration of hypotonic solution of Tris-HCl (50 mM, 100 mM & 150 mM). Left panel shows the image under UV and right panel shows the image under bright field.
a) shows the image of seminiferous tubule treated with linearized pCX-EGFP plasmid suspended in 50 mM Tris-HCl solution.
b) shows the image of seminiferous tubule treated with linearized pCX-EGFP plasmid suspended in 100 mM Tris-HCl solution.
c) shows the image of seminiferous tubule treated with linearized pCX-EGFP plasmid suspended in 150 mM Tris-HCl solution.

FIG. 7 depicts Testis observed under UV stereo zoom Microscope 30 days after interstitial injection with linearized pCX-EGFP construct suspended in various concentration of Tris-HCl (hypotonic solution):
a) shows image of testis in phase contrast administered with linearized pCX-EGFP suspended in 20 mM Tris-HCl.
b) shows image of testis under UV filter administered with linearized pCX-EGFP suspended in 20 mM Tris-HCl.
c) shows image of testis in phase contrast administered with linearized pCX-EGFP suspended in 60 mM Tris-HCL
d) shows image of testis under UV filter administered with linearized pCX-EGFP suspended in 60 mM Tris-HCL
e) shows image of testis in phase contrast administered with linearized pCX-EGFP suspended in 80 mM Tris-HCL
f) shows image of testis under UV filter administered with linearized pCX-EGFP suspended in 80 mM Tris-HCL
g) shows image of testis in phase contrast administered with linearized pCX-EGFP suspended in 100 mM Tris-HCL
h) shows image of testis under UV filter administered with linearized pCX-EGFP suspended in 100 mM Tris-HCl.
i) shows image of testis in phase contrast administered with linearized pCX-EGFP suspended in 150 mM Tris-HCl.
j) shows image of testis under UV filter administered with linearized pCX-EGFP suspended in 150 mM Tris-HCl.

FIG. 8 depicts immuno-histochemical analysis of testis after 30 days of administration with desired poly nucleotide fragments of pCX-EGFP constructs suspended in hypotonic solution of 150 mM Tris-HCl.
a) shows image under phase contrast.
b) shows image under TRITC filter.
c) shows image under FITC filter.
d) show merged image.

FIG. 9 shows hematoxylin and eosin staining of 150 mM Tris-HCl treated testis showing intactness of spermatogenesis.

FIG. 10 shows PCR analysis of genomic DNA depicting propagation of inserted transgene (MIS-IRES2-EGFP) through SSC to next generation

Lanes 5-29: MT 1-MT 25, Lanes 32-42: MT 26-MT 36, Lanes 48-66: MT 42-MT 60, Lanes 2-4, 43-45, 67: gDNA of WT mice, Lanes 1, 46, 68: no template, Lanes 31, 47, 69: plasmid DNA.
MT denotes transgenic animal of MIS-IRES2-EGFP line from FVB/J strain. WT denotes wild type mice. MG—denotes the MIS-IRES2-EGFP fore-founder mice from FVB/J strain.
MIS=Promoter of Sertoli cell specific gene of Mullerian Inhibiting Substances.

FIG. 11 depicts Slot blot analysis of the PCR positive progenies obtained from the fore founder males MG 1, MG 2 and founder animals MT 5 and MT 21.
MT—denotes transgenic animal of MIS-IRES2-EGFP line from FVB/J strain.
WT—denotes wild type mice.
MG—denotes the MIS-IRES2-EGFP fore-founder mice from FVB/J strain.

FIGS. 12(a to f) show expression of GFP in the Sertoli cells of 7 days old transgenic mice as compared to age matched WT mice.
a) shows image under UV FITC filter.
b) shows corresponding merge image (under DIC and FITC filter) of (a).
c) shows enlarged view of semeniferous tubules of (a) under UV FITC filter.
d) shows corresponding merge image (under DIC and FITC filter) of (c).
e) shows image of wild type mice testis section under UV FITC filter.
f) corresponding merge image (under DIC and FITC filter) of (e).

FIG. 13 show western blot analysis for GFP expression in tissue extracts of the testis of the transgenic mice carrying MIS-IRES2-EGFP transgene introduced by the method of the invention.
a) protein isolated from 7 day old testis of transgenic as well as wild-type mice were used to detect EGFP protein through Western Blot. Two of the three transgenic mice showed GFP expression (˜28 kDa band), which was absent in the protein samples from wild-type mice.
b) transblot was probed with β-actin antibody which was observed in all the samples.

### DETAILED DESCRIPTION OF THE INVENTION

There is a need for an easily adaptable, universal (for all animals) and efficient method of transgenesis. In view of the various drawbacks associated with the available techniques, transgenesis cannot be practiced in every laboratory desiring to have such animals. The inventors of the present invention address this issue by establishing a rapid and robust method of transgenesis using Spermatogonial Stem Cells (SSCs). Spermatogonial Stem Cells (SSCs) are unique among adult tissue stem cells because its genotype is passed through the germ line to subsequent generations. SSC have the ability to self-renew and to produce progenitor cells (type A spermatogonia) that give rise to the entire spermatogenic lineage.

Russell et al. (In Histological and Histopathological Evaluation of the Testis (Russell, L. D., Ettlin, R. A., Sinha Hikim, A. P., & Clegg, E. D., eds, 1990 pp. 1-40, Cache River Press, Clearwater, Fla.) have stated that spermatogenesis is among the most productive of self-renewing systems with approximately 12 amplifying divisions between the stem cell and the differentiated product. Therefore, once committed to spermatogenesis, a single rat SSC can theoretically produce 4096 mature spermatozoa and theoretically gene integration in one SSC can presumably produce 4096 genetically modified transgenic sperm. These transgenic sperm can also be used for producing transgenic animal. The present invention is more advanced than available techniques in all aspects-such as generation of several transgenic lines (with differential gene integration in different SSC of the same testis) with stable integration of gene of interest in their germ line within a very short span of time. Being a non-surgical and rapid method, the present invention also positively addresses ethical issues related to pain, suffering and mortality of the animals, involved in procedures described before, making it a unique and ethically superior technique. Further, the present invention may be actually used in easily generating genetically modified large/farm animal of commercial importance for using them as a bioreactor for production of biopharmaceuticals and nutraceuticals or disease model or elite and/or disease resistance strain. It may also be used as a germ line and/or somatic gene therapy approach to address the various diseases along with infertility.

### DEFINITIONS

Hypotonic Solution—a solution which has a lower osmotic pressure than any other solution (confined in a membrane structure viz. cell)

Non Surgical—any procedure that does not involve surgery.

Testes—the male sex gland in the scrotum in which sperm and testosterone are produced. Pair of testis is present behind the penis in a pouch of skin called scrotum.

Non-Human Vertebrate—all vertebrates except human beings like guinea pig, rabbit, rat, dog etc.

DNA—Deoxyribonucleic acid (DNA) it constitutes the primary genetic material of all cellular material, it occurs predominantly in the nucleus.

Spermatogenesis—this process includes all of the nuclear and cytoplasmic changes that transform the primordial germ cells of the male germ line into mature spermatocytes. The formation of mature sperm in the male testes after the onset of puberty.

Seminiferous Tubules—seminiferous tubules are located in the testes, and are the specific location of meiosis, and the subsequent creation of gametes, namely spermatozoa.

Genes—a length of DNA that carries the genetic information necessary for production of a protein. Genes are located on chromosomes and are the basic units of heredity.

Promoter—is a controlling element in the expression of the gene. It serves as a recognition signal for an RNA polymerase and marks the site of initiation of transcription. A promoter is a region of DNA that facilitates the transcription of a particular gene.

Knock out—excision or inactivation or deletion of a gene within an intact organism or even animal model usually carried out by a method involving homologous recombination.

Knock down—suppression of the expression of a gene product, typically achieved by the use of antisense oligo deoxynucleotides and RNAi that specifically target the RNA product of the gene. Gene knock down refers to techniques by which the expression of one or more of an organism's genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knock down organism”.

shRNA—small hairpin RNA (shRNA) contains sense and antisense sequences from a target gene connected by a loop, and is expressed in mammalian cells from a vector by a pol III-type promoter. The shRNA is transported from the nucleus into the cytoplasm, where Dicer processes it. Small hairpin RNA is expressed from a DNA template and processed into small RNAs to guide RNAi-mediated targeted mRNA degradation.

Wild type—the normal, typical phenotype of any mammal before genetic mutation takes place. Wild type (or wild type) refers to the phenotype of the typical form of a species as it occurs in nature.

Transgenic animal—a transgenic animal is one that carries a foreign gene that has been deliberately inserted into its genome. The foreign gene is constructed using recombinant DNA methodology.

Transgenic lines—generations of offspring produced by mating of transgenic models.

F0 Generation/Fore-founder—FVB/J male in which the transgene was injected in the testis was considered as Fore-founder.

F1 Generation—produced by mating of fore-founder male with the wild type female.

F2 Generation—produced by mating of F1 generation siblings positive for transgene integration.

Primer—is a strand of oligonucleotides that serves as a starting point for DNA replication.

PCR—a technique in which a region of a nucleic acid fragment is amplified using polymerase enzymes by the help of a specific primers.

Antibody—also known as immunoglobulins are gamma globulin proteins that are found in blood or other bodily fluids of vertebrates, and are used by the immune system to identify and neutralize foreign objects, such as bacteria and viruses. Molecules produced by B cells in response to specific proteins (antigens) carried by infected cells.

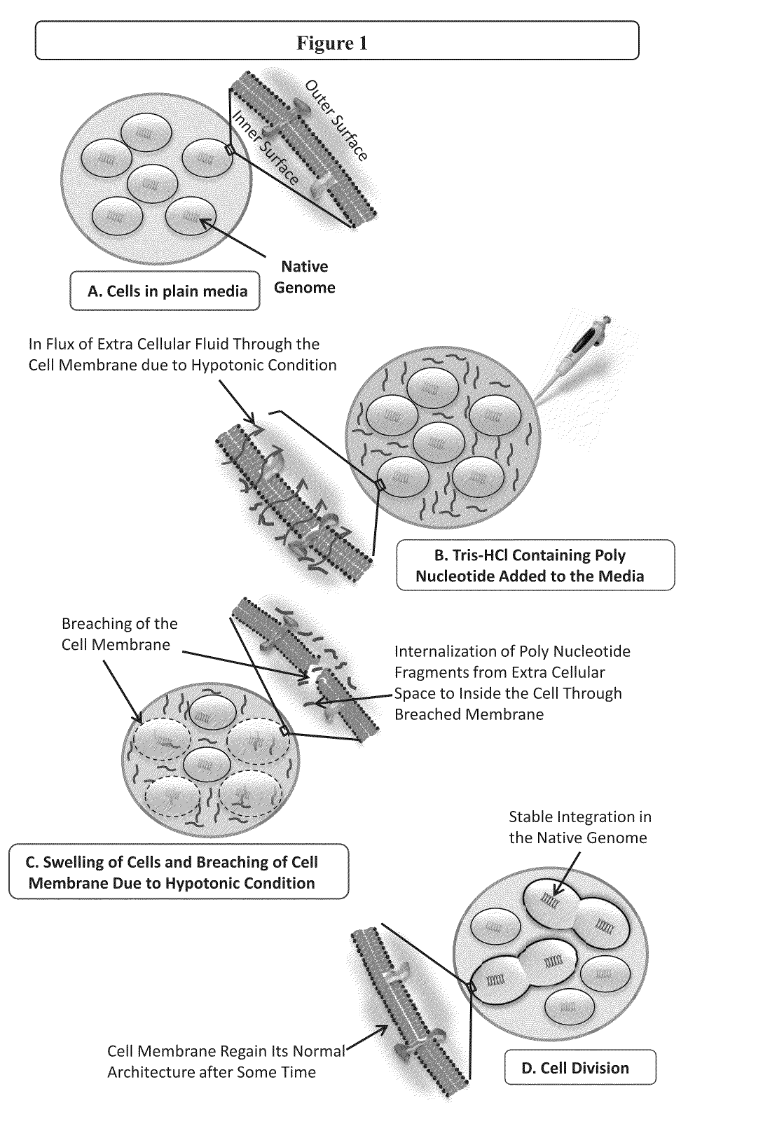

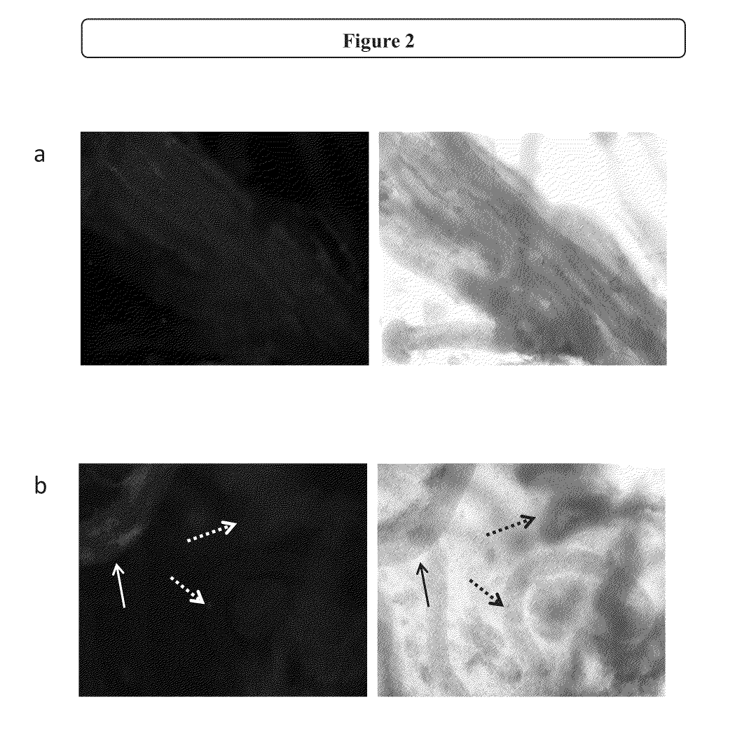

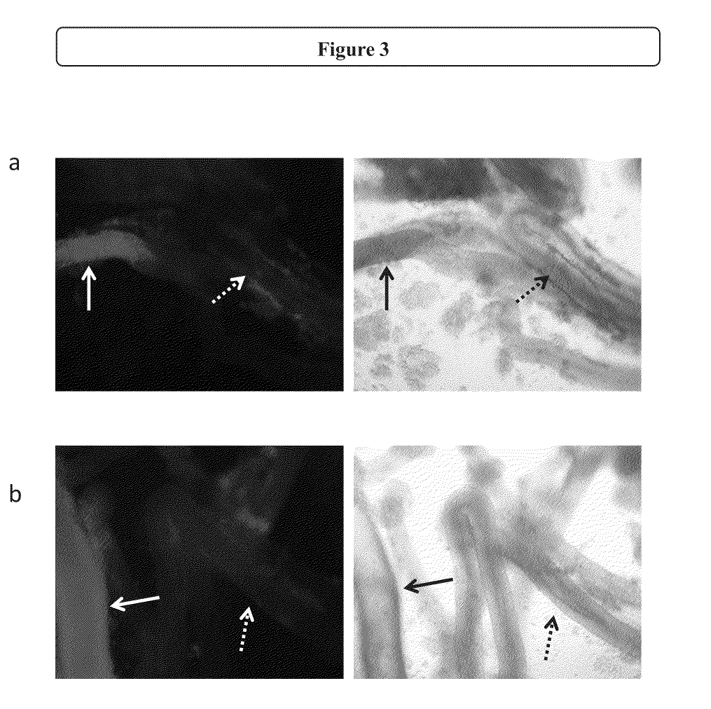

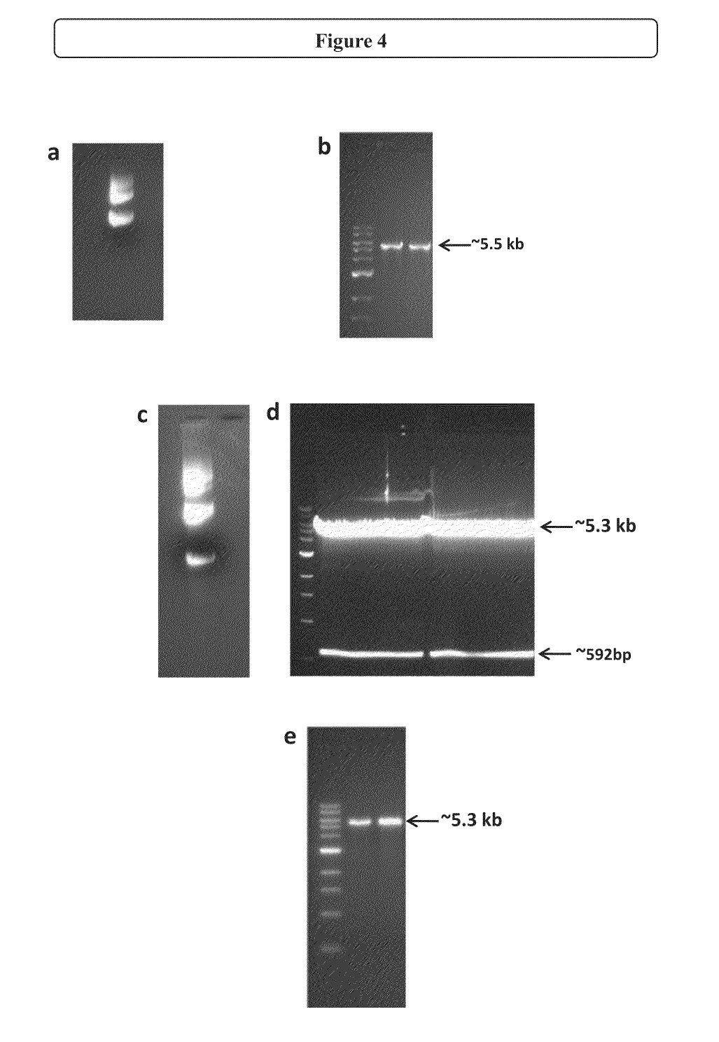

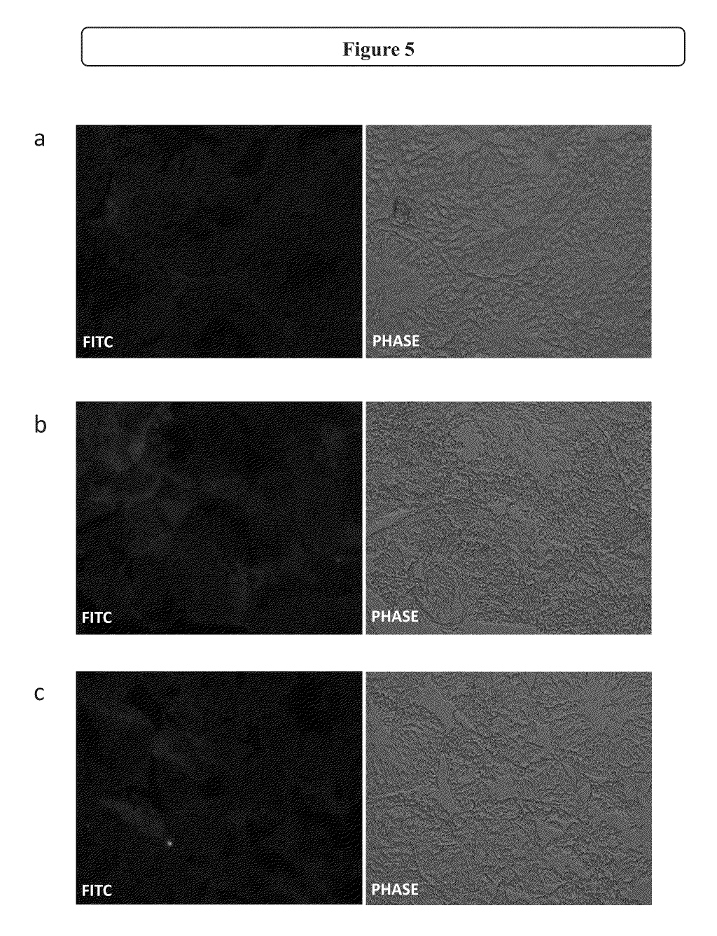

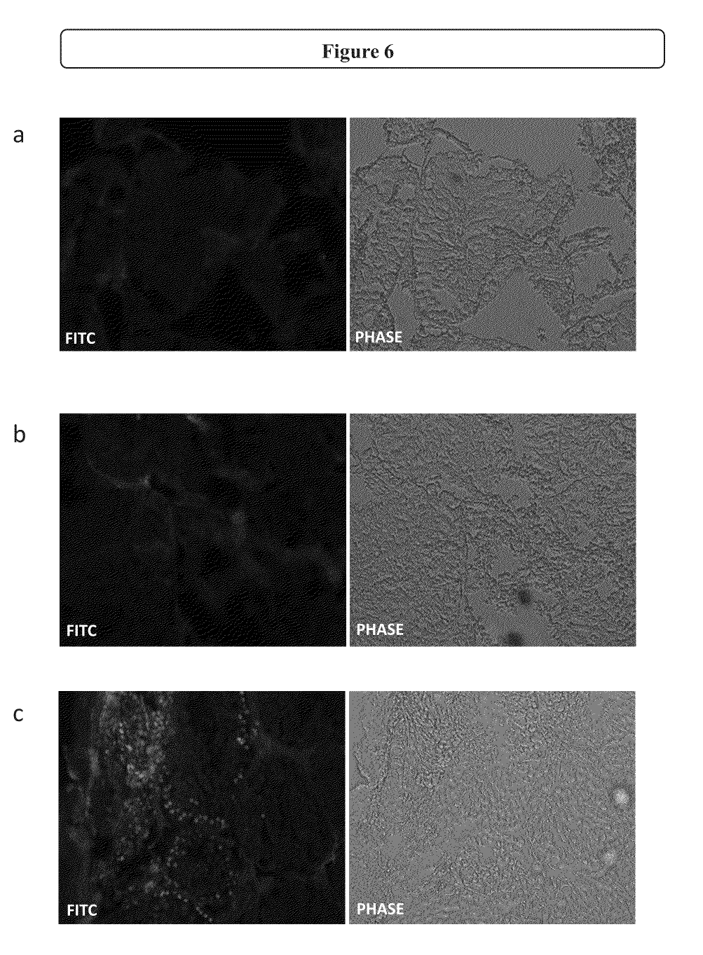

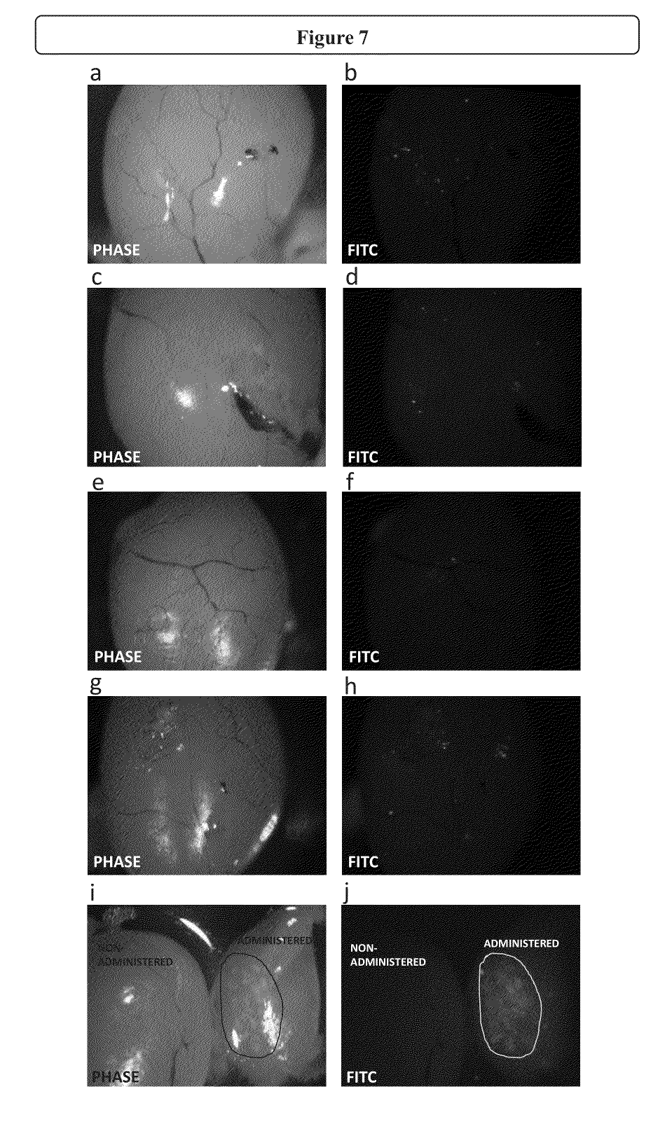

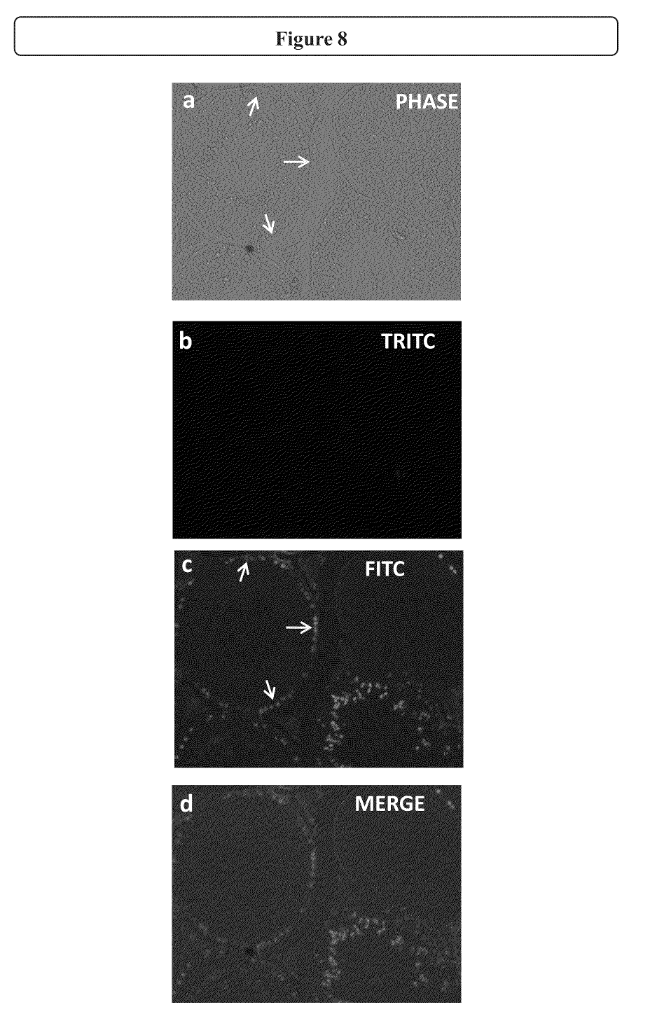

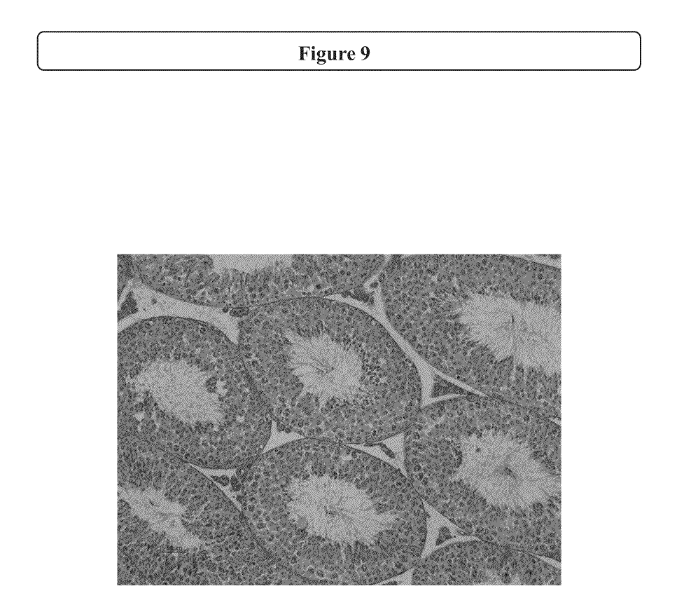

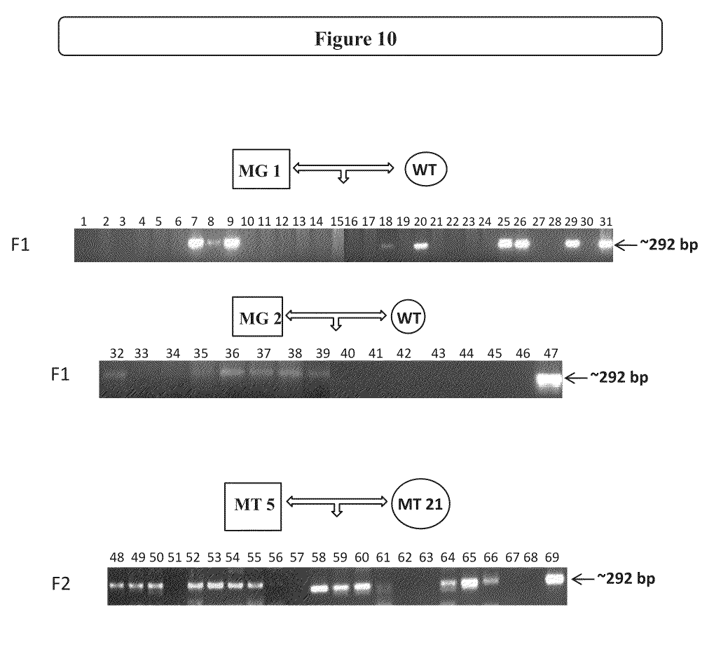

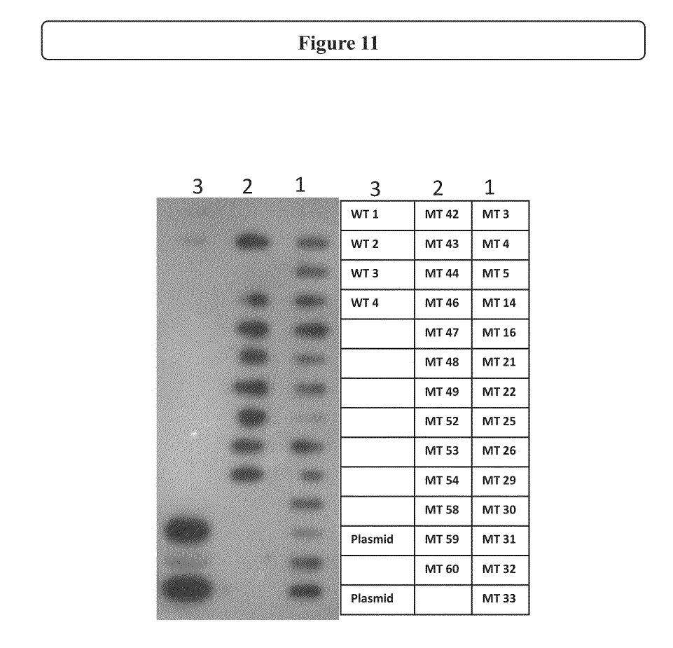

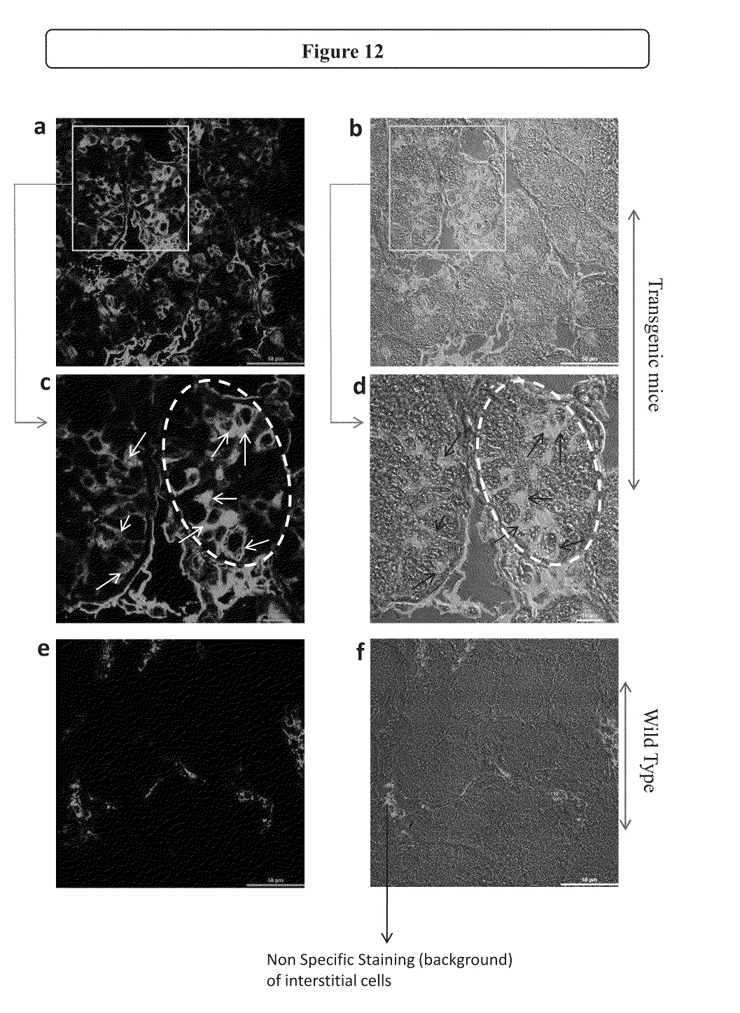

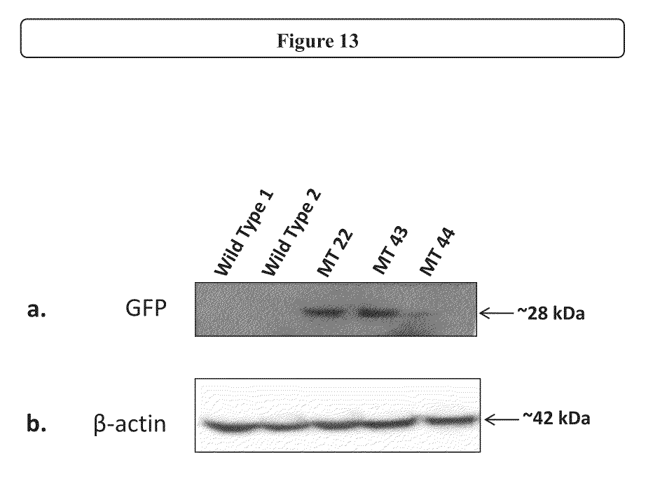

The present invention provides a simple and effective method of transgenesis. The method of the invention comprises use of hypotonic solution as a vehicle for transfer of poly nucleotide fragments into germ cells. In this method, short term exposure of germ cells to this solution in vitro or injecting DNA suspended in hypotonic solution into the testis of mice is carried out. Sperelakis, (Essentials of Membrane Biophysics. Academic Press. 2011, pp. 288. ISBN 978-0-12-387738-3) has stated that when a cell is exposed to a milieu of hypotonic solution the water will be drawn out of the solution into the cell by osmosis. If water molecules continue to diffuse into the cell, it will cause the cell to swell up to the point where cytolysis may occur. Based on this principle, the inventors hypothesized that maintaining hypotonicity surrounding the germ cell in a controlled manner can lead the cell to swell gradually exerting cytoplasmic pressure on the cell membrane causing transient breaching of the cell membrane. Once the cell membrane is temporarily breached, it allows internalization of the suspended poly nucleotide fragments inside the cell (FIG. 1). Once internalized, the poly nucleotide fragments get integrated into the native genome or regulate cellular process epigenetically. These cells then divide and proliferate through normal cellular process and generate sperm which carries the polynucleotide of interest and have potential to generate transgenic offspring if utilized. The hypotonic solution of Tris-HCl in water, adjusted to pH 7.0 causes temporary opening up of vertebrate male germ cell's membrane and facilitates delivery of nucleic acid fragments or transgene or gene of interest or gene as a therapeutic agent. Even though invention has been demonstrated with 150 mM Tris-HCl as the hypotonic solution, the molarity of hypotonic solution may vary depending on the type of cell membrane to be breached which can be inferred by a person skilled in the art.

The method includes administration of the hypotonic solution along with fragments of poly nucleotides which in turn consists of a promoter and a gene, into testicular tissues of vertebrate male in-vivo. These animals were kept for long term to detect and analyze the transfected and genome modified cells, utilizing the feature of the reporter gene concept.

The present invention provides a rapid procedure for integration of transgene in vertebrate male germ cells which can express the gene in an episomal manner and can also get integrated into native genome permanently. Even though the method of the present invention has been demonstrated using male germ cells, the method may be applied for other types of cells of vertebrate male testes, female germ cells and somatic cells also by altering the concentration of the composition.

Sperm generated from such germ cells can potentially generate transgenic animals. This is achieved by delivery of desired poly nucleotide fragments (DNA) in germ cells through cell membrane which is breached due to hypotonic treatment/shock. The procedure developed is very simple, cost effective and robust that facilitates gene integration in the testicular germ cells for generation of transgenic male germ cells. The method of generating transgenic animals using the sperm generated from the germ cells carrying the transgene is well known to a person skilled in the art.

In one embodiment of the invention, the method is a shortcut procedure of transgene integration by hypotonic shock to male germinal cells for gene expression and transgenesis comprising the steps of: isolation and purification of desired poly nucleotide fragments; preparation of a suspension of desired poly nucleotide fragments in 150 mM Tris-HCl of neutral pH; administration of said suspension into the vertebrate testis or testicular tissue; maintenance of the testis or testicular tissue in its normal physiological condition; detection and/or utilization of germ cells which have up taken the desired poly nucleotide fragments for desired objectives. Use of this method for delivering drugs or other therapeutic agents which do not diffuse through the cell membrane can also be envisaged by a person skilled in the art.

The invention is now described with reference to the following examples. The examples are described for the purposes of illustration and are not intended to limit the scope of the invention. Although specific embodiments of the present invention will now be described with reference to the drawings, it should be understood that such embodiments are by way of example only and merely illustrative of but a small number of many possible embodiments which can represent applications of the principles of the present procedural/process invention.

### Example 1

### In-Vitro Delivery of Red-dUTP Nucleotide Across the Testicular Seminiferous Tubules Based on the Principle of Hypotonicity after Isolating and Culturing them In Vitro Using Tris-HCl Solutions of Varied Concentration

Seminiferous tubules of the 30 days old FVB mice testis were exposed to red dUTP nucleotide (fluorophore labelled) suspended in different concentration of Tris-HCl hypotonic solution (50 mM, 100 mM, and 150 mM). After 10 minutes, tubules were washed thrice with 1×PBS buffer and recultured for 2 hrs. Such treated tubules were observed under UV after 2 hours of treatment for the presence of fluorescence inside the seminiferous tubules. It was observed that in tubules which were treated with 150 mM Tris-HCl hypotonic solution, Red-dUTP had moved substantially inside the tubules to greater extent as compared to the one which were treated with 100 mM Tris-HCl hypotonic solution whereas 50 mM Tris-HCl hypotonic solution treated seminiferous tubule did not show any integration fluorescent (Red-dUTP) nucleotide (FIGS. 2 and 3).

### Example 2

### Construct 1 Carrying EGFP as Reporter Gene, pCX-EGFP

Enhanced Green fluorescent protein (EGFP) gene cloned under the chicken beta actin promoter was used for the standardization of procedure. The chicken beta actin promoter in pCX-EGFP plasmid regulates the expression of EGFP gene (enhanced GFP gene—a variant of endogenous GFP gene of species Aequorea victoria) and make it available to fluoresce in all the tissue types ubiquitously. An enhancer region of human cytomegalo virus further strengthens its expression.

### Construct 2 Carrying EGFP as Reporter Gene, MIS-IRES2-EGFP

The EGFP in this plasmid is driven by the Mullerian inhibiting substance (MIS) promoter and is designed to express EGFP specifically in Sertoli cells of the testis because MIS is a Sertoli cell specific protein of testis found during infancy only.

### Example 3

### Isolation and Purification of Plasmid DNA from Bacterial Culture

Overnight grown bacterial culture was harvested and plasmid DNA was isolated using alkaline lysis method. A small fraction of isolated plasmid was checked by agarose gel electrophoresis and quantified by measuring optical density at 260 nm (FIGS. 4a and 4c). This was further processed for all the procedure.

### Example 4

Restriction Digestion, Elution and Purification of Plasmid DNA

Restriction digestion of both the construct 1 and construct 2 were performed as per supplier's instructions using Restriction Endonuclease Enzymes to linearize the constructs (FIG. 4b, 4d). These digested products were then purified either by gel elution method or by direct ethanol precipitation process as applicable. A small fraction of these purified products were then further checked by agarose gel electrophoresis (FIGS. 4b and 4e) and quantified by measuring optical density at 260 nm.

### Example 5

### In-Vitro Gene Delivery in to Testicular Germ Cells Based on the Principle of Hypotonicity by Using Tris-HCl Solutions of Varied Concentrations

Seminiferous tubules of the FVB mice testis were isolated and treated with suspension of linearized pCX-EGFP plasmid in various concentration of Tris-HCl hypotonic solution (2.5 mM, 5 mM, 10 mM, 50 mM, 100 mM and 150 mM). Duration of treatment was 10 minutes. Tubules were washed twice with 1×PBS and maintained in culture in presence of Follicle stimulating hormone (FSH) and Testosterone for 18 days with periodic media changes. After which the tubules were fixed in Bouin's solution and processed for immuno-histochemistry for detection of EGFP in germ cells. It was observed that seminiferous tubules which were treated with plasmid suspended in 150 mM Tris-HCl got transfected with the pCX-EGFP polynucleotide and showed EGFP fluorescence in spermatogonial cells whereas no such fluorescence was observed in tubules treated with other concentration of Tris-HCl hypotonic solution (FIGS. 5 and 6).

### Example 6

### In-Vivo Gene Delivery in to Testicular Germ Cells Based on the Principle of Hypotonicity by Using Tris-HCl Solutions of Varied Concentration Based on In Vitro Data

This was undertaken based on the results of in vitro gene transfection in male germ cells of seminiferous tubule. FVB/J male mice of 30±2 age were anesthetized by intra-peritonial injection of ketamine hydrochloride and xylocaine hydrochloride (45 mg/kg ketamine+8 mg/kg xylazine). Hair from lower abdominal and scrotal region was trimmed or removed and cleaned with savlon and betadine. After two minutes, sterile water was spread on scrotal area to remove excess betadine and make the area moist. Plasmid DNA suspended in various concentrations of Tris-Hcl (20 mM, 60 mM, 80 mM, 100 mM, 150 mM) containing 0.04% Trypan blue (optional and used to monitor the accuracy of the injection only) was injected slowly into the testis using 100 Hamilton syringe. A 0.5 inch long 26 gauge sterile needle (tuberculin syringe needle) was slowly inserted up to middle of the testis and removed before introducing tip of 100 Hamilton syringe (26 gauge) [701N; Hamilton Bonaduz A G, Switzerland] at the same site. This was done to facilitate the entry of the needle of Hamilton syringe which is long (2 inch) and hence tends to bend during insertion failing to overcome the resistance posed by the scrotal sac. 20-25 μl of the desired plasmid DNA (0.5 μg/μl) was delivered into the testis. In this procedure, DNA was injected in both testes.

### Example 7

### Observation of EGFP Expression In-Vivo Under UV-Stereo Zoom Microscope

The testicular tissue or whole testes were observed under stereo zoom microscope with UV filters to detect in-vivo EGFP expression. Testis injected with linearized fragment of pCX-EGFP suspended in various concentration of Tris-HCl (20 mM, 60 mM, 80 mM, 100 mM, 150 mM) were removed 30 Days post injection and observed under stereo zoom microscope. Testis administered with 20 mM, 60 mM, 80 mM, 100 mM, did not show any fluorescence under UV filter whereas testis administered with 150 mM Tris-HCl showed EGFP fluorescence (FIG. 7). The circle (black and yellow) in FIGS. 7(i) and 7(j) specifies the area that expressed EGFP under UV filter. The observation of fluorescence in germ cells in-vivo under microscope showed the expression of EGFP after 18 days or more (here after 30 days) which is confirmation of stable integration and expression of the transgene (here EGFP) in the genome of the vertebrate male germ cells.

### Example 8

### Tissue Fixation and Block Making

After dissecting the tissue, it was fixed in Bouin's solution (750 ml of picric acid, saturated aqueous solution, 250 ml of 37-40% formalin, 50 ml of glacial acetic acid) for overnight. The fixed tissue was dehydrated (ethanol series 50%, 70% and 100%) and embedded in paraffin wax using standard protocol.

### Example 9

### Immuno-Histochemical Analysis of Tissue Sections

The sections of various tissues were cut (3-5μ) using microtome (Reichert Jung, USA) and deparaffinized before processing for immuno-histochemical analysis. The sections were incubated with primary antibody diluted in 1×PBS containing 1% BSA in a moist chamber for overnight. This was then followed by three washings in 1×PBS for 5 min each. The secondary antibody was also diluted in similar way and section was incubated with secondary antibody in a similar fashion for 5 h. The section was washed three times in 1×PBS for 5 min each. The section was mounted in ProLong Gold antifade reagent (Molecular Probes, Invitrogen, USA) and was observed under fluorescence microscope (Nikon, Japan) with suitable filters. To stain for GFP protein, primary antibody used was mouse monoclonal anti-GFP antibody (clontech, USA) at a dilution of 1:250 and the secondary antibody was goat anti-mouse IgG conjugated with Alexa fluor 488 (Molecular Probes, Invitrogen, USA) at a dilution of 1:250. Immuno-histochemical analysis for detection of GFP expression in fore-founder's testis (administered with 150 mM Tris-HCl along with desired fragments of PCX-EGFP constructs) revealed expression of EGFP in the germ cells after 30 Days (FIG. 8). White arrow marks the germ cell population. This data proves that present procedure is specific for this cell type and therefore delivers poly nucleotide fragments of interest in germ cells only. Presence of EGFP expression even after 30 days of transfection supported the hypothesis that integration of poly nucleotide fragments occur in the genome of SSCs by this procedure and the expression was not episomal. GFP expression in testis of 7 days old transgenic mice carrying MIS-IRES2-EGFP Poly Nucleotide Fragments was detected upon staining with the GFP antibody. GFP expression was observed specifically in Sertoli cells of the testis (arrows, Black and White). Aged matched wild type mice testis showed no EGFP expression (FIG. 12).

### Example 10

### Hematoxylin and Eosin Staining of Treated Testis

Hematoxylin and eosin staining of the testis after 30 days of treatment with 150 mM Tris-HCl along with poly nucleotide fragments did not show any disturbance in the testicular architecture thus proving the safety of the method (FIG. 9).

### Example 11

### Propagation of Transgene

FVB/J male in which the transgene was injected in the testis was considered as F0 (fore-founder) because it carried the gene in the germ cells of the testis. These fore-founders were cohabitated with wild type females (ratio, 1 male: 2 females) after 35 days of gene injection. Pups born were screened by PCR using genomic DNA obtained from their tail biopsies. Those positive for the transgene were regarded as F1 generation of transgenic animals. F2 generations of mice were generated by breeding PCR positive males and females from F1 generation. Pups born were analyzed to check whether the transgene is stably integrated and propagated to consecutive generation.

### Example 12

### Isolation of Genomic (g) DNA from Progeny

Tail biopsies from 3 weeks old pups were used for isolation of genomic DNA by standard phenol chloroform iso-amyl alcohol protocol.

### Example 13

### Propagation of the Transgene: Detection of Transgene by PCR Method

Genomic DNA was used for Polymerase Chain Reaction (PCR) using standard protocol (Mullis, 1990). The reaction mixture was denatured at 94° C. for 4 min. This was followed by amplification which was carried out for 35 cycles of denaturation at 94° C. for 45 sec. This was then followed by primer annealing (details of which are given in Table 2) for 45 sec and extension at 72° C. for 45 sec. Thereafter, a final extension at 72° C. for 10 min was carried out. The PCR products were then analyzed on TAE agarose gel. In order to rule out the possibility of false positives in the PCR reaction, negative controls such as PCR reaction with gDNA of wild type FVB mice and reaction without DNA template were also performed. For positive control, PCR reaction was carried out with the plasmid.

TABLE 2
Primer sequences used for genotyping of the
transgenic lines

| | | | | Pro- |
|---|---|---|---|---|
| | | | | duct |
| S. | | | Tₐₙₙₑₐₗ | Size |
| No. | Constructs | Primer Sequence 5′→3′ | (° C.) | (bp) |
| 1 | MIS-IRES2- | F: AAGCCCTTTGAGACAGTCGC | 62 | 292 |
| | EGFP | (SEQ ID NO: 1) | | |
| | | R: ATATAGACAAACGCACACCG | | |
| | | (SEQ ID NO: 2) | | |

PCR genotyping of the offspring using genomic DNA (gDNA) obtained from post weaning tail biopsies of progeny generated from fore founder MG 1 & MG 2 (FVB/J strain) administered with 150 mM Tris-HCl along with MIS-IRES2-EGFP poly nucleotide fragment. MG 1 & MG 2 were mated with wild type female mice of same strain (FVB/J). In the analysis, animal No. MT 3, 4, 5, 14, 16, 21, 22, 25, 26, 29, 30, 31, 32, and 33 shows a band of amplified product at position ˜292 bp. F2 progeny were generated by mating PCR positive males and females of F1 generation (MT 5 & MT 21). In F2 generation, animal Nos. 42, 43, 44, 46, 47, 48, 49, 50, 52, 53, 54, 58, 59, 60 showed a band of amplified product at position ˜292 bp hence proved to be positive (FIG. 10). These results prove that permanent integration and propagation of the desired poly nucleotide fragment is possible through this procedure.

### Example 14

### Propagation of the Transgene: Detection by Slot Blot Analysis

The results of PCR were further confirmed by Slot blot analysis (Lavigueur A. et al., 1989) of PCR-positive animals from each generation using radioactive labelled probe directed towards IRES2-EGFP.

The hybridization signals were detected by autoradiography. The samples, which were blotted on the membrane for hybridization, are MT 3-MT 33 (F1 generation pups of MG 1 & MG 2); MT 42-MT 60 (F2 generation pups of MT5 and MT21); WT1-WT4 gDNA from four different wild type mice. MIS-IRES2-EGFP fragment was used as positive control. Out of these samples, 6 samples (MT 3, MT 42, MT 44, MT 60, MT 59, and MT 58) showed no hybridization hence declared as false positive in PCR reaction. Rest of the samples proved to be positive in comparison with wild type samples which showed no hybridization as well (FIG. 11). IRES2-EGFP fragment isolated from MIS-IRES2-EGFP plasmid by restriction digestion were used for probe preparation.

### Example 15

### Propagation of the Transgene: Detection of Expressed Protein by Western Blot Analysis

Testis tissue samples were homogenized in 10-volumes of ice cold tissue lysis buffer [50 mM Tris-HCl (pH7.6), 150 mM NaCl, 1% Triton X-100]. The samples were then centrifuged at 14000 g for 15 min at 4° C. and supernatant was collected and stored at −20° C. Protein estimation was carried out using Bradford reagent (Sigma-Aldrich, USA) following manufacturer's instructions. Sodium dodecyl sulphate-poly acrylamide gel electrophoresis (SDS-PAGE) was carried out according to the principle described by Shapiro (Shapiro et al., 1967). Separated proteins were electro-transferred to PVDF membrane using Tris-Glycine buffer (25 mM Tris, 192 mM glycine) containing 20% methanol. Following blocking, membranes were incubated with primary antibodies diluted in blocking solution at concentrations recommended by manufacturers for overnight at 4° C. After three washes of 5 min each with TBS-T at room temperature, membranes were incubated with appropriate dilution of HRP conjugated antibody, diluted in the blocking solution for 1 h at room temperature. The membranes were washed with TBS-T at room temperature (5 washes for 5 min each) and detection was done using enhanced chemiluminescence method (ECL, Amersham Biosciences, UK). Testicular tissue of PCR positive 7 days old animal, generated through breeding of treated Fore Founder (administered 150 mM Tris-HCl along with MIS-IRES2-EGFP polynucleotide fragment) with Wild Type female, were used for protein isolation along with age matched control. This shows the presence of EGFP in the positive animal and absence of the EGFP in their control counterpart (FIG. 13). This result led to the conclusion that permanent genomic integration and propagation of the desired poly nucleotide fragment is possible through this procedure.

The present procedure is novel, user friendly and fast, causing breaching of germ cell membrane and delivery of poly nucleotide fragments. It also provides a method for stable genomic integration (in genome inside nucleus), which include integration of transgene or shRNA, or site specific knock-in and/or knock-out of the desired nucleotide fragments or epigenetic (in cytoplasm) modification using RNAi in the male germ cells. The gene integration by this procedure withstands the process of mitosis and meiosis in testis and generates transgenic sperm which can be further used for generating transgenic non human vertebrate models and could be potentially used for propagating gene for therapeutic protein production by farm animals. Present procedure can also be used to facilitate gene therapy. If gene integration in any stem cells hampers cell division i.e. mitosis and meiosis—it would not yield any sperm thereby acting as a self regulated filter.

More specifically, the invention provides a method for the permanent gene integration in SSC. The invention also provides a non surgical method for generating a large number of transgenic animals from a single fore founder male in short span of time. The term “large number” not only emphasizes the proven possibility of this invention to generate huge pool of transgenic or genome modified animal in terms of number but also in terms of copy number variation which may differ from SSC to SSC generating respective sperm within the same testis. The invention provides an ethically superior procedure by using the endogenous physiological process of the body to propagate the transgene avoiding the requirement of any animal sacrifice for obtaining testicular germ cells or superovulated oocytes. The invention also envisages provision for generating large transgenic animals like cattle and monkey which at present is difficult and cumbersome to develop using presently available techniques.

### ADVANTAGES OF THE INVENTION

simple, rapid, economical and cost effective
a short term exposure to described hypotonic solution is enough for gene delivery in germ cells
non-surgical and efficient way of in vitro and in vivo gene delivery
can be utilized for generating transgenic animals without involvement of assisted reproductive techniques or specialized laboratory setup
does not require highly trained personnel hence is user friendly and easily adaptable.
allows stable integration of transgenes into the genome of germ cells

Although certain embodiments of the invention are described herein in detail, it will be understood by those skilled in the art that variations may be made thereto without departing from the spirit of the invention or the scope of the appended claims.

We claim:

1. A method of gene integration in cells comprising the steps of:
a. preparation of a suspension of poly nucleotide fragments comprising the desired gene in a hypotonic solution;
b. administration of the suspension of step (a) to cells or tissues; and
c. maintenance of the cells or tissues in vitro or in its normal physiological condition.

2. The method as claimed in claim 1 wherein said polynucleotide comprises of deoxyribonucleic acid (DNA), ribonucleic acid (RNA), small hairpin ribonucleic acid (shRNA), small interfering RNA (siRNA), microRNA (miRNA), long non-coding RNA.

3. The method as claimed in claim 1 wherein said hypotonic solution of step (a) is Tris-hydrochloride (Tris-HCl) solution.

4. The method as claimed in claim 3 wherein said Tris-HCl solution has molarity of 150 mM at neutral pH.

5. The method as claimed in claim 1 wherein said cells comprises germ cells, other cell types of the vertebrate male testis, and somatic cells.

6. The method as claimed in claim 5 wherein said germ cells comprises male and female germ cells.

7. The method as claimed in claim 6 wherein said male germ cell is Spermatogonial Stem Cells (SSC) or advanced germ cells of seminiferous tubule of the testis of the FVB mice.

8. The method as claimed in claim 1 wherein the poly nucleotide integrated into genome is inheritable to the next generation.

9. A method of generating a transgenic animal comprising the steps:
a) preparation of a suspension of desired poly nucleotide fragments in a hypotonic solution;
b) administration of the suspension of step (a) to germ cells or tissues;
c) maintenance of the germ cells or tissues in vitro or in its normal physiological condition; and
d) utilization of sperm generated from germ cells containing the desired polynucleotide fragments in natural mating or assisted reproductive technique (ART) to generate transgenic animals.

10. The method as claimed in claim 9 wherein said ART comprises in vitro fertilization (IVF) and intra-cytoplasmic sperm injection (ICSI).

11. The method as claimed in claim 9 wherein said poly nucleotide fragments is stably integrated in the native genome.

12. The method as claimed in claim 11 wherein said stably integrated poly nucleotide is inherited to the next generation through the germ line.

13. Use of the method as claimed in claim 1 for generation of transgenic male germ cells in vitro and/or in vivo for making transgenic animals as a bioreactor for production of biopharmaceuticals and/or nutraceuticals or in gene therapy in addition to generating humanized disease models for research and development.

14. Use of the method as claimed in claim 9 for generation of knock-out animals or generation of knockdown animals or generation of knock-in animal or in gene therapy for permanently removing a bad trait that occurs due to gene malfunction or gene mutation.


SEQUENCE LISTING (2 sequences)

SEQ ID NO: 1 - DNA, 20 bp, Artificial Sequence
  A synthetic primer
aagccctttgagacagtcgc

SEQ ID NO: 2 - DNA, 20 bp, Artificial Sequence
  A synthetic primer
atatagacaaacgcacaccg